(19) United States
(12) Patent Application Publication
(10) Pub. No.: US 2004/0048359 A1
(43) Pub. Date: Mar. 11, 2004

(54) Test strips moveable by magnetic fields

(76) Inventor: William R. Schmeling, Middletown, DE (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/619,091

(22) Filed: Jul. 14, 2003

Related U.S. Application Data
(60) Provisional application No. 60/395,459, filed on Jul. 12, 2002.

(51) Int. Cl. C12M 1/34

(52) U.S. Cl. 435/287.1

(57) ABSTRACT

Test strips, kits containing the tests strips, and methods for making, moving, sorting, storing, immobilizing, or otherwise handling test strips are disclosed herein. The test strips are used in material analyses and include a magnetically attractive material disposed upon or within the test strips in such an amount and at such a location that the test strips will move or will adopt a desired spatial orientation or will align in response to a magnetic field.

### CROSS-REFERENCE TO RELATED APPLICATIONS

Priority is claimed to U.S. Provisional Application No. 60/395,459, filed Jul. 12, 2002, which is incorporated by reference herein.

### FIELD OF THE INVENTION

The present invention relates generally to test strips, test kits containing test strips, methods of making test strips and test kits, and methods of moving, sorting, storing, immobilizing, or otherwise handling test strips.

### BACKGROUND OF THE INVENTION

Test strips are devices used for chemical or other analyses of samples or compositions. Test strips typically include a base material (also referred to as a backing material), most commonly a fibrous web or other porous structure. Many test strips also contain additional components such as pads of material for collecting or wicking solutions, filters, membranes, and substrates having desired compositions or characteristics. Many such components provide locations for conjugates or reactants to interact with the sample or means for allowing samples to travel along a base material. Test strips further contain one or more substances that will cause the test strips to exhibit observable effects in response to one or more properties of a material being tested. For example, some test strips exhibit a response to materials that contain a specific analyte. In some embodiments, the substance that causes the strip to display the effect is the material that forms the base material itself, the material that forms other components of the test strip, or a substance contained within or attached upon the base material or other components.

Many test strips contain a base material that will allow a sample to flow through components of the test strip by capillary action, gravity, or other means. In many test strips, samples flow via capillary action through structural components attached to the base material (e.g. pads, nitrocellulose membranes, etc.). This type of test strip is used most commonly with samples present in a liquid phase, although test strips are used to analyze samples in any phase or combination of phases. Many test strips used for liquid samples have one or more zones that contain different substances, porous components, membranes, filters, or combinations of the foregoing through which a sample is drawn as it moves through the components of the test strip. Such strips are frequently used to test samples for the presence, amount, concentration, or characteristics of a specific analyte that is itself a liquid or is dissolved, suspended, dispersed, or otherwise contained within a liquid. Analytes in the sample interact with the substances contained within the different zones on the test strip as the sample traverses the length of the strip. The presence, absence, amount, or characteristics of specified analytes will affect whether a signal is generated on the strip as well as the type and strength of the signal. A signal commonly used with test strips is the appearance or change of color on the test strip or a portion thereof. Such strips are commonly referred to as chromatogenic test strips. Other types of signals can be used, generally depending on the analyte and substances contained in the test strips. Examples include, but are not limited to, luminescence and radioactivity. The test reagents contained within the different regions and components of the test strip will vary depending on the type of sample involved, the analytes for which testing occurs, and the type of signal being used for detection. Test strips are commonly packaged in kits that provide the user with instructions for testing conditions and procedures.

For many test strips, the substances are distributed heterogeneously on the strip such that the reactant distribution is not symmetrical on the strip in one or more axes or directions. For example, some test strips contain substances at only one end, in which case it is important to assure that the sample is contacted with that end of the test strip. As another example, obtaining an accurate test result for some test strips depends on assuring that the sample contacts different substances on or components of the test strip in the proper order. Such strips will not function properly unless the sample (typically a liquid sample) moves in the correct direction along the strip. For these types of strips, it is often necessary to assure that the sample is applied or contacted at the correct location on the strip. Since many test strips are rectangular, this requirement usually means applying the sample at the correct end or edge of the strip. When assembling a test kit, it is desirable to ensure that strips of this type are oriented in the proper direction in the kit. Furthermore, some test strips, though substantially flat in shape, will have one or more portions that are thicker than or shaped differently from other portions of the strip. When such strips are to be packaged in inflexible packaging having a specific shape, some strips need to be aligned in a specific direction when packaged.

A common method for manufacturing test strips involves first applying the appropriate reactants and other components to portions of paper or other base material that are larger than test strips, commonly referred to as “cards.” The cards are then cut into individual strips. In many cases, a single card is cut into a large number of strips. A common example results in an average of approximately 100 strips per card, although processes exist in which a single card results in greater or fewer numbers of strips. Some of the strips may be rejected due to imperfections in card sub-components, and the ends of the cards are typically discarded.

A problem with current test strip manufacture is the absence of an expeditious method for orienting cut strips in the proper direction. For example, upon exiting the cutting processes, strips often are conveyed or fall into collection containers without control over the resting orientation of the strip. For test strips having heterogeneous distributions of substances, this often results in a different orientation for each strip in a group of collected cut strips. The varied orientation of cut strips makes it necessary to sort and to align the strips. In many cases, single production lots contain tens of thousands of strips. In many existing processes, the sorting and alignment of cut strips is performed by hand, an expensive and laborious process. Thus, it would be desirable to develop a faster, less labor intensive way to orient and to align test strips after cutting.

A related problem with test strip manufacture relates to counting the appropriate number of test strips for preparation of packages or batches of a desired number. After cutting, the test strips in a lot are often transferred to containers for storage or shipment. At some point between manufacture and sale, the strips are often counted into groups of a specific number of test strips prior to sale. The number is typically much smaller than the number of strips in a lot. In one example, a lot containing thousands of cut strips is transferred to containers each holding 50 test strips. Thus, the strips must be counted into batches of the appropriate size for each container. Counting and sorting the strips by hand is a slow, labor-intensive process. It is therefore desirable to find a means for automatic test strips counting.

Another problem with test strip manufacture relates to separating test strips that are considered to not meet specification or are defective, damaged, stained, or otherwise undesirable from batches of acceptable test strips. Cards that are processed to result in numerous acceptable test strips may contain regions having defects or other properties making those regions of the card undesirable for use in test strips. Examples include, but are not limited to, surface irregularities and marks. Such regions will be referred to as “aberrant regions” throughout this application and test strips containing aberrant regions or portions thereof will be referred to as “aberrant test strips” throughout this application. Aberrant regions can result from any cause, including but not limited to errors in processing the cards and defects or irregularities in the raw materials used to form cards. In many processes, aberrant regions on cards are marked (for example, with a colored marking pen) prior to cutting so that aberrant test strips can be identified and removed after cutting. Strips containing aberrant regions are removed after marking because aberrant regions do not necessarily occur at the ends of the card and thus are not easily removed without cutting areas in the middle of the card. The result of this process, however, is a large batch containing a mixture of what is typically a very large number acceptable test strips and typically a small number of aberrant test strips. Inspecting the batch to identify and to remove aberrant test strips typically involves visual inspection of the entire batch, and thus is a labor-intensive process. It is therefore desirable to identify ways of reducing the labor and time needed to separate aberrant test strips from acceptable test strips.

### SUMMARY OF THE INVENTION

The present invention solves the problems in the prior art by reducing the labor required to orient, to count, and to sort the cut test strips during or after manufacture. Specifically, test strips are described herein having a magnetically attractive material located at one or more locations on the strip. The magnetically attractive material is present in an amount and location or locations effective to cause each strip to move or to align or orient itself spatially in response to the magnetic field.

The invention further includes pluralities of test strips, wherein the pluralities include a first group including one or more of the test strips having a magnetically attractive material located at one or more locations on the strips as described in the preceding paragraph, and a second group including one or more of the test strips that do not contain magnetically attractive material present in an amount effective to cause the second group of strips to move or to align or orient themselves spatially in response to the magnetic field.

The invention further includes kits for conducting analysis of materials wherein the kits contain magnetically attractive test strips.

The present invention further includes methods of making test strips that can be moved, aligned, or oriented through application of a magnetic field. The methods produces strips that have a magnetically attractive material at substantially the same location or locations on each strip. The method includes incorporation of a magnetically attractive material into each strip. The magnetically attractive material must be present in a sufficient amount such that the strip will move or change alignment or orientation in response to a magnetic field. In a preferred embodiment, the method includes forming a card, applying the magnetically attractive material to one end of the card prior to cutting, then cutting the card into strips such that a portion of the magnetically attractive material is located at the same end on each strip. In some embodiments, the method includes preparing test strips such that some test strips contain magnetically attractive material, while others do not. One way of accomplishing this is removing the magnetically attractive material from certain test strips or from the portions of the card that will result in those test strips. The material is preferably removed from the cards prior to cutting the card. Removing the magnetically attractive material from those areas of the cards allows use of a magnetic field to separate test strips into two groups based on whether or not they include magnetic material. In a preferred embodiment, the magnetically attractive material is removed from the portion of cards that contain aberrant regions or that will result in aberrant test strips. In this embodiment, the magnetic field is used to separate aberrant test strips from acceptable test strips.

The present invention also includes methods of counting, aligning, handling, sorting, storing, inventorying, or transporting test strips wherein the method includes providing a test strip containing a magnetically attractive material and using a magnetic field to move test strips, to align or orient the test strips, to assure that the test strips retain a spatial alignment or orientation, or to immobilize test strips in a desired location or orientation or combinations of the foregoing. In some embodiments, the method involves a group including one or more test strips that contain a magnetically attractive material and another group that includes one or more test strips that do not contain a magnetically attractive material, and a magnetic field is used to separate the two groups from each other. In a preferred embodiment, the group that does not possess magnetically attractive material includes aberrant test strips.

Accordingly, it is an object of the present invention to provide test strips that will move or change their spatial orientation or alignment when desired.

It is a further object of the present invention to provide kits containing test strips that move or change special orientation when desired.

It is a further object of the present invention to provide methods of making test strips that move or change their spatial orientation or alignment when desired.

It is a further object of the present invention to provide methods of moving or spatially orienting or aligning test strips.

It is a further object of the present invention to provide methods of counting test strips, such as counting for the purpose of determining the total number of test strips in a batch, counting to assist in dividing a batch into groups of defined quantities, or both.

It is a further object of the present invention to provide methods of handling, sorting, separating, transporting, or storing test strips, or combinations of these activities.

These and other objects, features and advantages of the present invention will become apparent after a review of the following detailed description of the disclosed embodiments and the appended drawings.

### BRIEF DESCRIPTION OF THE FIGURES

FIG. 1 depicts an example of one embodiment of a test strip of the present invention.

FIG. 2 depicts one embodiment of a process for making test strips of the present invention in Panels A, B, C, D, and E.

FIG. 3 depicts another embodiment of a process for making test strips of the present invention in Panels A, B, C, D, E and F.

### DETAILED DESCRIPTION

Test strips containing magnetically attractive material in effective amounts to cause the test strips to align, orient or move in response to a magnetic field, text kits containing such test strips, and pluralities of test strips in which some test strips contain an effective amount of magnetically attractive material and other test strips do not contain an effective amount of magnetically attractive material are provided. Methods of making the test strips described herein as well as methods of handling, sorting, separating, transporting, storing, or performing a combination of these activities with respect to test strips are also provided. In a preferred embodiment, the test strips containing magnetically attractive material are rectangular test strips that contain magnetically attractive material at or near one end of the test strip. A preferred magnetically attractive material is a metal tape that is applied to test strips or cards from which the test strips are made by placing the metal tape between two components located at or near that end.

Throughout this application, the term “aberrant region” shall mean a region on a card that contains defects or other properties making those regions of the card undesirable for use in test strips. Examples include, but are not limited to, surface irregularities, the presence of too much or too little of a component of a test strip, and marks. Test strips containing aberrant regions or portions thereof will be referred to as “aberrant test strips” throughout this application.

Test Strips

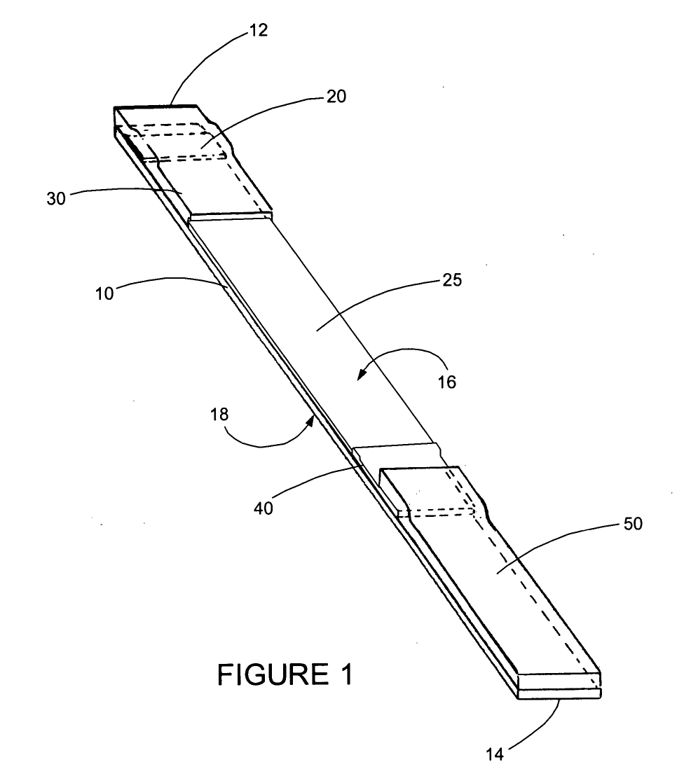

The term “test strips” refers to articles that are used for chemical or other analysis of materials, samples or compositions. Test strips of the present invention include any object containing a substance or group of substances that will exhibit an observable response to one or more properties if present in a material tested. The response is any type including but not limited to signals such as the existence or degree of a color change, luminescence, radioactivity, or emission or reflectance of electromagnetic radiation, including electromagnetic radiation of one or more specific wavelengths. A preferred response is the color change characteristic of chromatogenic strips. The strips can have or include any shape, whether symmetrical or asymmetrical, regular or irregular. Examples of some preferred regular shapes include square, circular, triangular, rhomboid, diamond, and rectangular. An especially preferred shape is a rectangular shape in which the long side of the rectangle is 5-10 times as long as the short side of the rectangle, the edges of the strip are the short side of the rectangle, and the portions of the strip closest to those edges are the “ends” of the strip. As used in this application, the term “strip” or “test strip” is not limited to shapes that are relatively flat or narrow in one dimension. However, strips that are substantially flat in one dimension are preferred. Strips that are substantially flat but have pads on each end, similar to that shown in FIG. 1, are especially preferred. Examples of test strips include, but are not limited to, those disclosed in U.S. Pat. No. 6,096,563 to Mapes.

Test strips described herein can thus be moved or aligned by applying a magnetic field to them. In preferred embodiments, the magnetically attractive material is located at substantially the same location on every strip, so that the magnetic field causes all of the strips to be oriented in the same direction. By “substantially the same location” it is meant that material is at a location that is sufficiently close to the same end or edge of each strip to assure that all of the strips, or nearly all of the strips, will align, move, orient or perform a combination of the foregoing, in the same direction upon application of a magnetic field to the strips.

Referring now to FIG. 1, in one embodiment the strip of material is substantially flat in one dimension such that it has two ends 12, 14 a top 16 and a bottom 18. The sample includes a base material 10 to which a magnetically attractive material 20 has been applied. A sink pad 30 has been applied on the same end as the magnetically attractive material 20 such that the sink pad 30 covers the magnetically attractive material 20 and the magnetically attractive material 20 is sandwiched between the base material 10 and the sink pad 30. A conjugate pad 40 is applied near the opposite end 14 of the test strip from the location to which the magnetically attractive material 20 was applied and a membrane 25 is disposed between the conjugate pad 40 and the sink pad 30 and overlaps the conjugate pad 40 and sink pad 30 to a sufficient extent to allow sample to flow between these components. A filter pad 50 is placed at the end 14 of the test strip that is opposite of the end 12 to which the magnetically attractive material 20 and sink pad 30 were applied. It will be appreciated that in this embodiment the magnetically attractive material 20, sink pad 30, conjugate pad 40, membrane 25 and filter pad 50 are all applied to the top side 16 of the base material 10. The filter pad 50 in this preferred embodiment is the location at which the sample is applied by dipping that end of the strip into the sample or otherwise contacting the strip with the sample. The sink pad 30 is located at the opposite end of the strip and receives the sample material after it has traveled the length of the strip. As the sample travels from the filter pad 50 toward the sink pad 30 it first passes through the conjugate pad 40 then through the membrane 25, and finally into the sink pad 30. In one embodiment, reactants are located in the conjugate pad 40 under conditions such that they will bind or react with one or more analytes, if such analytes are present in the sample. Additional zones not shown containing further conjugate or reactant exist along the membrane 25 at locations not shown between the conjugate pad 40 and the sink pad 30. In some preferred embodiments, the passage through the conjugate pad 40 followed by the membrane 25 results in the formation of one or more signals on the membrane 25 indicating one or more characteristics regarding the sample and its constituents. It is to be understood that the foregoing is only one embodiment of a test strip and that the invention includes all types of test strips.

As used throughout this application, the terms “base material,” “backing material,” and “base component” shall refer to the component of a test strip that provides structural support for other test strip components and may also provide a means for analytes or samples to move between and through other components of a test strip. Although the term “base material” is used here as a convenience to describe a component of certain embodiments of this invention, it is to be understood that the invention is not limited embodiments in which the base material performs any of the functions listed in this definition and is not limited to embodiments in which a specified structural base material is found.

The base material of the strip include any material or substance that will allow the sample to flow along its desired path and will allow any substances in the test strip to exhibit their desired response. Examples include, but are not limited to, solid materials formed by extrusion or shaping, paper or cellulose webs and webs of fibers made of or containing synthetic polymers such as polystyrene. A preferred base material is made of polystyrene fibers. A preferred material for the membrane is nitrocellulose and preferred material for the pads include polyester fibers and cellulose fibers.

Sample characteristics detected by test strips include any characteristic that is determined by the presence or absence of a response. In a preferred embodiment, the characteristic is the presence, absence, amount, or concentration of one or more specific analyte. As used herein, the term “analyte” refers to any material or substance for which the presence or amount can be determined. Examples include but are not limited to any compounds, molecules, or objects including, without limitation, organic or inorganic chemicals or compounds of any size and type, prokaryotic or eukaryotic cells, cell fragments, cell debris, fragments of cell walls, organelles and other cell components, viruses, and fragments of viral walls and components of any type of protistan, moneran, plant, animal, or fungal organism including but not limited to components that can be derived (for example, by extraction) from such organisms prior to detection. Some preferred compounds, molecules or chemicals include, but are not limited to, nucleic acids, amino acids, carbohydrates, peptides, polypeptides, proteins, lipids, hormones, growth factors, neurotransmitters, immunoglobulins, monokines and other cytokines, antibodies, proteoglycans, glycoproteins, glycosaminoglycans, glycolipids, lipoproteins, salts, metals, minerals, ions, aromatic hydrocarbons, chlorinated hydrocarbons (including, without limitation, perchloroethylene, trichloroethylene, polychlorinated biphenyls, dioxins, and furans), nitroaromatics, pesticides, herbicides, and fungicides.

The substance or substances that cause the strip to exhibit a response is the one or more components of the test strip (e.g. pads, membrane or base material) or substances contained within or upon such component. Substances are distributed homogeneously or heterogeneously on or within the base material. In a preferred embodiment, one or more substances are distributed heterogeneously on the strip. In an especially preferred embodiment, several substances are distributed in individual patterns such that a liquid sample flowing along the strip will contact each substance in a specific desired order.

The strips of the present invention contain a magnetically attractive material. The magnetically attractive material is added to the strips by any means, include attachment thereto, deposition thereon or encapsulation or containment therein. The magnetically attractive material is applied to or incorporated into the strip before, during, or after manufacture of the strip. The magnetically attractive material is present in an amount effective to cause the strip to move or to align or orient itself in response to a magnetic field. In preferred embodiments, the magnetically attractive material will cause the alignment, orientation, or motion of the strip even in response to a relatively weak magnetic field. In some embodiments, the strips will move, orient or align when within a distance of approximately three inches or less from a magnet with a maximum pull rating of approximately 0.33 pounds or more. In other embodiments, the strips will move, orient or align when within a distance of approximately three inches or less from a magnet with a maximum pull rating of approximately 0.63 pounds or more. In other embodiments, the strips will move, orient or align when within a distance of approximately three inches or less from a magnet with a maximum pull rating of approximately 4 pounds or more.

The magnetically attractive material of the present invention includes any materials that will move in response to a magnetic field. The magnetically attractive material should be present in an amount effective to cause the strip to move or to change in orientation or alignment in response to a magnetic field. Desirable examples include, but are not limited to, materials containing ferrite, babingtonite, chromite, columbite, ferbite, franklinite, ilmenite, iron-nickel, magnetite, maghemite, manganbabingtonite, platinum, pyrrhotite, siderite, tantalite, and hematite. In a preferred embodiment the magnetically attractive material utilized in the system is a strip of metal or metal ore containing iron.

The magnetically attractive material is applied to the test strip in any form that will exhibit the desired response. Examples include, but are not limited to coatings or saturants that are applied wet, then dried to leave the magnetically attractive material in place, and solids that are affixed to the strips by means such as adhesives. In one preferred embodiment, the magnetically attractive material is a strip of metal or metallic foil affixed with an adhesive material on one side, preferably a pressure sensitive adhesive. In a further preferred embodiment, the magnetically attractive material is a strip of metallic foil affixed to one end of a rectangular strip.

The invention further includes pluralities of test strips that include a first group of one or more test strips that contain magnetically attractive material in effective amounts to cause the test strips to align, orient or move in response to a magnetic field and a second group of one or more test strips that do not contain an effective amount of magnetically attractive material. The second group includes test strips in any way including, but not limited to, test strips from which the magnetically attractive material has been removed, test strips that did not previously contain magnetically attractive material, test strips prepared from the same card as some of the test strips in the first group but from portions of the card that did not contain magnetically attractive material or from which magnetically attractive material had been removed. In some embodiments, the second group of test strips is prepared from the same card or cards as the first group of test strips but were prepared from portions of the card or cards from which the magnetically attractive material is removed prior to cutting the cards into test strips. In a preferred embodiment, the second group of test strips include aberrant test strips or test strips that contain portions of what were aberrant regions on the cards. The two groups may be separated from each other by application of a magnetic field to the plurality of test strips.

Test Kits

The kits provided herein contain the test strips described herein along with any other reagents, materials or substances useful in performing the desired analysis or performing other desirable activities with the kits. In some embodiments, the kits include reagents to be applied to the strip. In some other embodiments, kits include simply the test strip and instructions for use. Some kits include, for example: multiple test strips, multiple reagents, or combinations thereof to allow multiple tests; containers for the test strips; additional accessories, or any combination of the foregoing. The kits may include any combination of one or more test strips described herein with literature, accessories or any other objects or combinations thereof. As a non-limiting example, some kits include color charts or other guides that assist the user in interpreting the results of the tests, for example by determining a positive or negative signal for the presence of one or more analytes in the sample being tested or investigated.

Methods of Making Test Strips

Any method can be used to make the test strips of the present invention, including any method for preparing a test strip known in the art and any methods that become known in the future. The only critical step of the method is the addition of the magnetically attractive material before, during, or after the manufacture of the test strips.

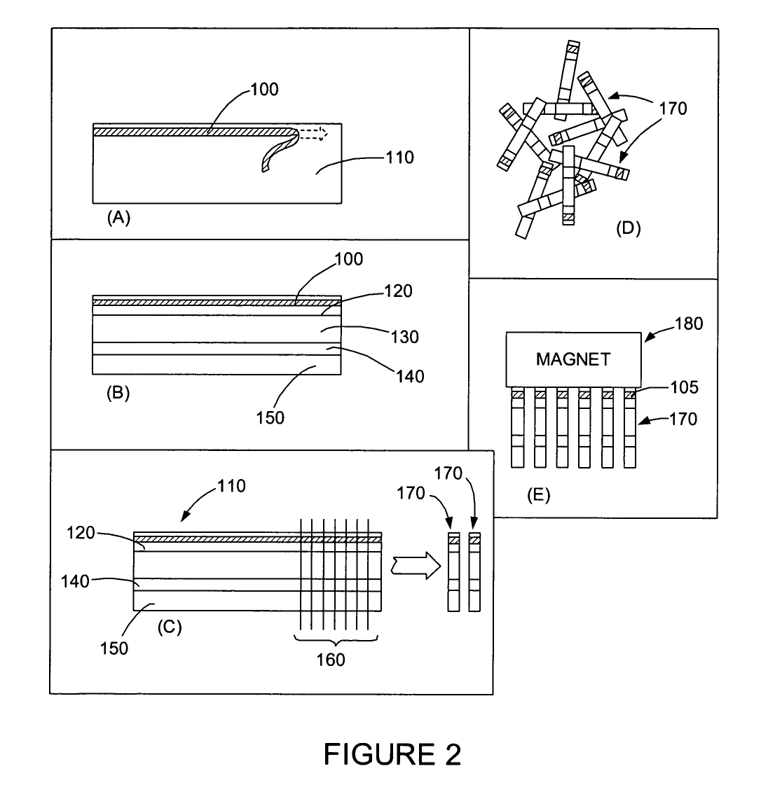

In a preferred embodiment, referring to FIG. 2, cards of Backing Material are prepared from base material. A length of magnetically attractive material is then applied along the edge of the cards as shown in panel A of FIG. 2. In panel A, magnetically attractive material, in this embodiment in the form of a flexible tape 100 containing iron, is applied to a card 110 of base material. The magnetically attractive material in this embodiment is applied to the edge that corresponds to the end of the strip that will contain the sink pad. Additional components are then added as layers to the card. In panel B, sink pad material 120 is applied to the base material such that it covers the tape, although in this view the portion of the sink pad material 120 that covers the tape 100 is cut away such that the location of the tape 100 can be seen. Membrane material 130, conjugate pad material 140 and filter pad material 150 are also applied such that the relative locations of these components are substantially identical to those locations shown on the test strip in FIG. 1. Proceeding to panel C, cards 110 are then sliced along axes 160 that are perpendicular to the axes in which the tape 100 and pads 120, 140, 150 cross the card 110 to result in cut strips 170. This slicing approach is preferred because it results in strips that each contain a portion of the tape and that have the same spatial distribution of components. In a preferred embodiment, cutting is performed with a MATRIX 2360 Programmable Shear available from Kinematic Automation, Inc. of Twain Harte, Calif. Several methods and devices for preparing cards of backing material, incorporating substances into the cards, and cutting cards into strips are known to persons of ordinary skill in the art. Any known or future methods or combination of such methods may be used. In the absence of some method to control the alignment of the strips after cutting, the cutting results in strips 170 aligned in a variety of directions as shown in panel D. In panel E, it is shown that placement of the strips 170 in proximity to a magnet 180 causes attraction of the portions of the strips 170 that contain the pieces of the tape 100 toward the magnet 180. The result is that the ends of the strips 170 that include the tape 105 are all attached to the magnet 180 and the strips 170 are all aligned along substantially the same axis.

The foregoing orders of steps in the described embodiments are not intended to be limiting. In other embodiments, the process order is changed. For example, in some embodiments the magnetically attractive material is applied at other points of the process, such as after the reagents and other components are applied, after cutting the cards, or at any other point before, during, or after the manufacturing process. Application of the magnetically attractive material before the reagents and other components are applied and before cutting is a preferred embodiment because it provides a way to cover the magnetically attractive material with other components and assures that the magnetically attractive material is in place before cutting, thus allowing the use of a magnetic field to sort the cut strips. There are also methods in which cards are not used because strips are prepared individually or are cut from different types of structures.

In some preferred embodiments, the method results in a plurality of test strips that includes a first group of one or more test strips that contain magnetically attractive material in effective amounts to cause the test strips to align, orient or move in response to a magnetic field and a second group of one or more test strips that do not contain an effective amount of magnetically attractive material. Examples of the test strips of the second groups include, but are not limited to, test strips that contain virtually no magnetically attractive material, test strips that contain a smaller amount of magnetically attractive material, and test strips that contain material that is sufficiently less magnetically attractive than the test Any method can be used to produce the two groups of test strips including, but not limited to: preparing the two groups from separate processes in which one process applies magnetically attractive material to test strips or cards and the other process does not; preparing the two groups from the same card or group of cards but removing parts of the magnetically attractive material from the cards prior to cutting into strips; and preparing the two groups from the same card or group of cards but applying the magnetically attractive material in a pattern or distribution such that only some of the test strips that result from cutting the cards will include the magnetically attractive material.

In one preferred embodiment, the method includes preparation of cards that contain magnetically attractive material, then removing portions of the magnetically attractive material from the portions of the card that will result in aberrant test strips.

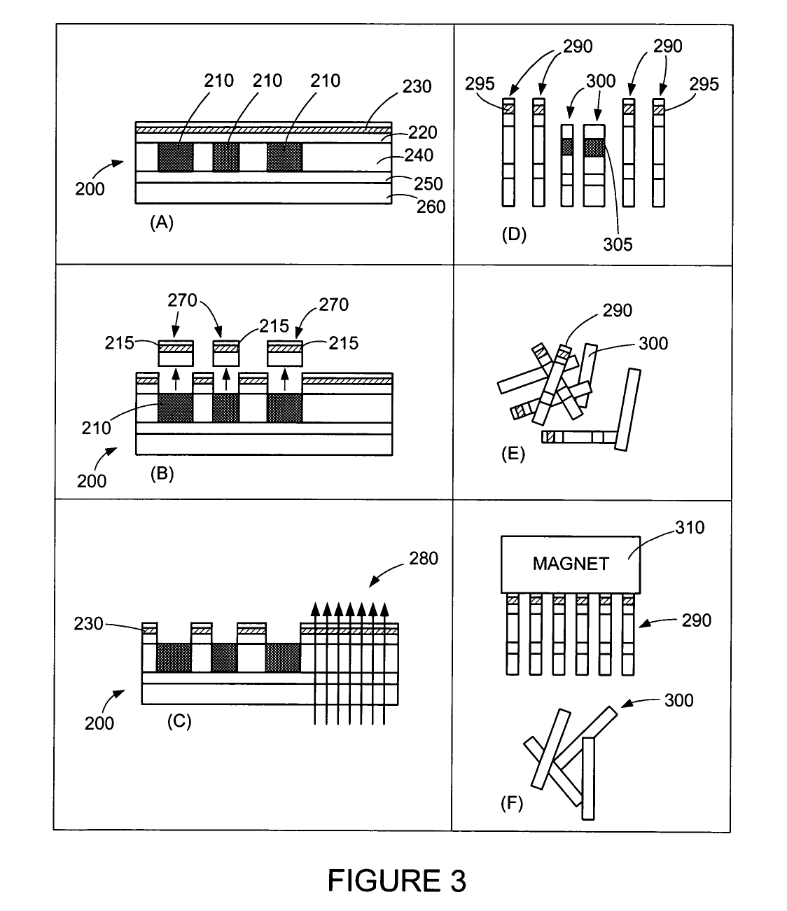

Referring now to FIG. 3, a card 200, is prepared as shown in panel A. The card includes sink pad material 220 with magnetically attractive material 230 located thereunder sink pad material 220 is partially cut away to show magnetically attractive material 230 in FIG. 3, membrane material 240, conjugate pad material 250, and a filter pad 260. These components of the card are prepared using substantially the same procedures described in connection with FIG. 2 above. Inspection of the card 200 reveals the existence of aberrant regions 210 on the card. Optionally, the aberrant regions 210 are marked with a pen or marker to make the areas more readily distinguishable by eye not shown. In panel B, portions 270 of the card 200 that contain magnetically attractive material 215 but that are located adjacent to the aberrant regions 210 are removed from the card 200 by excising the portions 270 out of the card 200. The portions 270 include parts of the magnetically attractive material 215 that, if not removed, would become part of the same strips as the aberrant portions 210 when the card 200 is later sliced into strips. In panel C the card 200 is then sliced along axes 280 that run in a direction that is perpendicular to the axis along which the magnetically attractive material 230 was applied to the card 200. The result of the slicing, shown in panel D, is two groups of test strips 290, 300. The first group includes strips 290 that include magnetically attractive material 295 but do not include aberrant regions or portions thereof. The second group of test strips 300 do not contain magnetically attractive material, but do contain aberrant regions or portions thereof 305. As with the test strips shown in FIG. 2, panel E of FIG. 3 shows the resulting jumbled and misaligned test strips that include strips 290 of the first group as well as strips 300 of the second group. In panel F, both groups of test strips are subjected to a magnetic field by placing a magnet 310 in close proximity to the combined groups of strips. The strips 290 of the first group are aligned and attracted to the magnet, while the strips 300 of the second group do not contain magnetically attractive material and thus are not attracted to the magnet. As a result, strips 290 from the first group, which do not contain aberrant regions, are separated from strips 300 of the second group that contain such regions.

Methods of Moving, Aligning, and Orienting Test Strips.

The invention also includes methods of moving, aligning, orienting, or performing any combination of the foregoing activities with respect to the test strips described herein. Moving includes any change in position or location. Orientation includes changes of the directional disposition of an object with respect to other objects, to its surroundings, or to a specific axis, especially for an object of heterogeneous shape or composition. An example of “orientation” is causing a specific end or part of the object to be positioned in a specific direction with respect to other parts of the object or to be “pointed” in a specific direction. Aligning test strips includes bringing a group of test strips into substantially the same orientation or into a desired pattern of orientations. The method includes application of a magnetic field to the test strips. The magnetically attractive material causes the strips to move, orient, align, or perform any combination of these activities in response to the magnetic field. In one preferred embodiment, a source of a magnetic field is placed in close proximity to the strips such that the strips are attracted toward and attach to the source of the magnetic field. In another embodiment, strips are placed in a space between two poles of a magnetic field and orient or align themselves in response to the field. All uses of magnetic fields are included.

The means by which the magnetic field is created and applied to the strips is not critical to the methods disclosed herein. Magnetic fields are applied through the use of a permanent magnet, an electromagnet, or any other means. In some embodiments, orientation is accomplished simply by passing a magnet over a bin containing strips that have been cut from a card. The source of the magnetic field is moved by any means, including by hand or by an automated process. In these embodiments the strips move toward the source and thus become closer to the source when applied. The strips need not actually contact the magnet. However, in one embodiment, the magnetic field causes the strips to adopt a specific orientation and to move vertically toward the magnet such that they become suspended from the magnet and such that the same end or edge of each strip is in contact with the magnet. The strips hanging from the magnet are then removed and packaged.

The invention includes use of magnets of any shape or composition and magnetic fields of any orientation. Fields are simple or complex in shape and orientation. In some embodiments, magnets or fields of irregular shapes or multiple fields are used to produce a specific desired pattern of orientation. Examples of simple magnet shapes include, but are not limited to, rods, bars, channel horseshoes, horseshoes, discs, blocks and rings. Examples of compositions of permanent magnets include, but are not limited to: alnico, ceramic, rare earth, and flexible (rubber) type magnets. Rubber type magnets may composed, for example, of neodymium, samarium-cobalt, strontium ferrite, aluminum-nickel-cobalt, copper, iron or titanium.

Other Methods of Processing and Handling Test Strips

The presence of magnetically attractive material on test strips allows numerous types of movement and control of test strip inventories. Such options are available to multiple persons throughout the chain of production, processing, and use including, for example, test strip manufacturers, personnel involved in sorting, packaging, storage, transportation and sales of test strips, and users of test strips. Any method of handling, transporting, moving, sorting, storing, immobilizing, or otherwise processing test strips using magnetic fields is contemplated herein within the present invention. In some embodiments, the presence of magnetically attractive material is used as a mechanism to move or to convey test strips using a magnetic field that moves or otherwise changes in field configuration. In some embodiments, magnetic fields are also used in storage. Magnetic fields are also used to sort, track, or otherwise organize strips. For example, magnetic fields are used to move strips into storage containers for shipment. In another embodiment, an end user uses a rack or other group of magnets arranged in a pattern (for example, a grid pattern) to sort test strips as they are used in the laboratory and labels or otherwise designates positions in the pattern in a way that identifies the tests and samples associated with the specific strips.

In some embodiments, a group of strips is counted and placed into smaller groups of a specific number. In one embodiment, the smaller groups are collected in containers. The containers are placed on a scale capable of measuring weight increases in the amount of a single strip. Although the weight of the strip is not limiting, in one embodiment the individual strips weigh between 0.17 grams and 0.23 grams each. Strips gathered and aligned with a magnet are dropped into the container. The weight increase measured by the container is used to determine the number of strips added. In another embodiment, a large group of strips is located on a scale calibrated to detect weight decrease in increments equal to the weight of a single strip. A magnetic field is then applied such that the field strength in the vicinity of the strips gradually increases and more strips are drawn toward the magnet. This is done, for example, by moving a permanent magnet closer to the strips, or by adjusting the electric current through an electromagnet. Monitoring the drop in weight allows counting the number of strips drawn out of the bin and toward the magnet during a pass and thus expedites the process of counting out batches of a desired size.

In other embodiments, test strips containing magnetically attractive material and test strips lacking magnetically attractive material or that contain less magnetically attractive material are sorted or separated from each other by applying a magnetic field to the test strips. The test strips that contain magnetically attractive material or that contain a greater amount of magnetically attractive material will respond to the magnetic field and the other group of test strips will not respond or will exhibit a weaker response. The groups are sorted from each other using the difference in their responses to the magnetic field. In some embodiments, a magnetic field attracts test strips containing magnetically attractive material and thereby separates those strips from strips that lack or contain less magnetically attractive material. In a preferred embodiment, the two groups are prepared from the same card or groups of cards and the strips lacking magnetically attractive material contain aberrant regions or portions thereof.

### EXAMPLE 1

Test strips having magnetically attractive material therein were prepared according to one embodiment of the present invention. Test strips having a width of 4 millimeters and a length of 83 millimeters were made of polystyrene. The strips were cut from cards having a width of 43 centimeters and a length of 83 millimeters. Prior to cutting, the cards were treated such that they possessed four different zones, each containing one or more substances through which an aqueous sample could travel by capillary action. The cards also had a strip of Steel Tape available from Innotec of Wisconsin, Inc., of Racine, Wis. The Steel Tape used was 0.125 inches in width. A 43-centimeter length of the Steel Tape was applied along a line close to the edge of card that would correspond to the sink end of the strips after cutting. Steel Tape is magnetically attractive because it contains iron in an amount sufficient to be attracted by magnetism. Steel Tape has a pressure sensitive acrylic adhesive on one side that allows it to be adhered to the test strip material. The zones of substances and the tape were applied to the cards along axes perpendicular to the line of cutting. Subsequent to the application of the substances and tape to the cards, the cards were cut into strips of the dimensions specified above using a MATRIX 2360 Programmable Shear available from Kinematic Automation, Inc. of Twain Harte, Calif. Ends of cards and strips with defective cuts were discarded. A cutting resulted in approximately 100-105 strips per card. Due to the perpendicular axis of cutting as compared to the axis along which the substances were applied, each test strip had a portion of Steel Tape along its sink end.

The strips were aligned by passing a permanent magnet over a bin containing the strips at a height of approximately three inches over the strips. The magnet attracted the Steel Tape and thus lifted the strips in an aligned fashion such that the sink end containing the Steel Tape attached itself to the magnet.

In a further step, approximately 39,000 strips were sorted into containers holding 52 strips each. A packaging container was placed on scale capable of detecting weight increase in increments of the weight of a single strip, and a funnel was positioned over the container such that strips dropped into the funnel would fall into the container. The magnet was passed over a bin containing the 39,000 strips in successive passes such that each pass picked up approximately 20-35 aligned strips. After each pass, the strips were collected from the magnet by hand and dropped into the funnel. The aligned strips fell through the funnel and landed in the container. The scale was used to measure the weight, and therefore the number, of strips dropped into the container after each pass so that the strips could be counted and it could be determined when 52 strips had been placed into the container.

All patents, publications and abstracts cited above are incorporated herein by reference in their entirety. It should be understood that the foregoing relates only to preferred embodiments of the present invention and that numerous modifications or alterations can be made therein without departing from the spirit and the scope of the present invention.

### EXAMPLE 2

Procedures essentially identical to those of EXAMPLE 1 are followed, except that the procedure further includes separating aberrant test strips from other test strips. Aberrant regions on the cards were first marked with a marker. Prior to cutting, magnetically attractive material was removed from the portions of the card that, upon cutting, would result in test strips that contained a portion of the aberrant regions (i.e., aberrant test strips). The aberrant test strips thus did not contain magnetically attractive material. As a result, the magnet did not pick up any of the aberrant test strips and the aberrant test strips remained in the bin and were not transferred to any of the packaging containers.

What is claimed is:

1. A test strip comprising a magnetically attractive material.

2. The test strip of claim 1, wherein the magnetically attractive material is present in the test strip in an amount and at one or more locations upon the test strip such that the test strip moves or adopts a specific spatial orientation or alignment when exposed to a magnetic field.

3. The test strip of claim 1, wherein:
the test strip is substantially flat and has a rectangular shape such that the test strip possesses two short edges of a first length and two long edges of a second length, the second length being longer than the first length; and
the magnetically attractive material is present in a zone on the test strip that is located such that the distance from the zone to one of the two short edges is shorter than the distance from the zone to the other of the two short edges.

4. The test strip of claim 1, wherein the magnetically attractive material is a tape affixed to the test strip, wherein the tape comprises iron.

5. A test kit comprising the test strip of claim 1.

6. A plurality of test strips comprising:
a first test strip that is the test strip of claim 1; and
a second test strip that comprises no magnetically attractive material or comprises a sufficiently less magnetically attractive material or a sufficiently smaller amount of magnetically attractive material than the first test strip such that:
the second test strip will not respond to a magnetic field to which the first test strip will move, align, or orient in response thereto, or
the second test strip will exhibit a response to a magnetic field that differs from the response of the first test strip thereto.

7. A method of sorting the plurality of test strips of claim 6, comprising:
applying a magnetic field to the plurality of test strips, and
separating the first test strip from the second test strip by use of the difference in the responses of the first test strip and the second test strip to the magnetic field.

8. The method of claim 7, wherein the difference in the responses of the first test strip and the second test strip to the specific magnetic field comprises movement of the first test strip in response to the specific magnetic field and less movement or no movement of the second test strip in response to the specific magnetic field.

9. A method of making the test strip of claim 1, comprising the following steps, in no particular order:
a) preparing the test strip from one or more components, and
b) incorporating the magnetically attractive material into the test strip.

10. The method of claim 9 wherein the magnetically attractive material is a tape comprising iron and (b) comprises affixing the tape to the cards.

11. The method of claim 9, wherein step a) comprises:
assembling cards from one or more components, and
dividing the cards into test strips.

12. The method of claim 9, wherein step b) occurs during step a).

13. The method of claim 12, wherein step a) comprises:
assembling cards from one or more components, and
dividing the cards into test strips.

14. The method of claim 12, wherein the method further comprises removing portions of the magnetically attractive material from the card prior to dividing the cards into test strips such that some of the test strips that result from the dividing do not comprise magnetically attractive material.

15. The method of claim 9 wherein the magnetically attractive material is a tape comprising iron and (b) comprises affixing the tape to the cards.

16. A method of aligning, moving, immobilizing, or orienting one or more test strips, wherein:
the one or more test strips are the test strip of claim 1, and
aligning, moving, immobilizing, or orienting the one or more test strips comprises exposing the test strips to a magnetic field.

17. The method of claim 16, wherein:
the one or more test strips are substantially flat and have a rectangular shape such that the test strips possess two short edges of a first length and two long edges of a second length, the second length being longer than the first length; and
the magnetically attractive material is present in a zone that is located on the one or more test strips such that the distance from the zone to one of the two short edges is shorter than the distance from the zone to the other of the two short edges.

18. A method of counting test strips wherein the test strips are the test strip of claim 1 and the method comprises:
applying a magnetic field to the test strips under such conditions as to cause the strips to move; and
counting the test strips as they move in response to the magnetic field.

19. The method of claim 19, wherein:
a) the test strips are located in a container prior to moving;
b) the strips exit the container when they move in response to the magnetic field; and
c) counting the test strips as they move in response to the magnetic field comprises monitoring changes in the gross weight of the container as the strips exit the container.

20. The method of claim 18, wherein:
a) the test strips are deposited into a container after the test strips move in response to the magnetic field;
b) counting the test strips as they move in response to the magnetic field comprises monitoring changes in the gross weight of the container as the strips exit the container.
